SEQUENCE LISTING (21 sequences)

SEQ ID NO: 1 - protein, 5 aa, Artificial
  Synthetic peptide
QYNAD

SEQ ID NO: 2 - protein, 5 aa, Artificial
  Synthetic peptide
QYNDA

SEQ ID NO: 3 - DNA, 15 bp, Artificial
  Synthetic probe
cartayaaygcngay

SEQ ID NO: 4 - DNA, 15 bp, Artificial
  Synthetic probe
cagtacaacgccgac

SEQ ID NO: 5 - protein, 5 aa, Artificial
  Synthetic peptide
NYNAD

SEQ ID NO: 6 - protein, 5 aa, Artificial
  Synthetic peptide
SYNAD

SEQ ID NO: 7 - protein, 5 aa, Artificial
  Synthetic peptide
TYNAD

SEQ ID NO: 8 - protein, 5 aa, Artificial
  Synthetic peptide
YYNAD

SEQ ID NO: 9 - protein, 5 aa, Artificial
  Synthetic peptide
QQNAD

SEQ ID NO: 10 - protein, 5 aa, Artificial
  Synthetic peptide
QNNAD

SEQ ID NO: 11 - protein, 5 aa, Artificial
  Synthetic peptide
QSNAD

SEQ ID NO: 12 - protein, 5 aa, Artificial
  Synthetic peptide
QTNAD

SEQ ID NO: 13 - protein, 5 aa, Artificial
  Synthetic peptide
QYQAD

SEQ ID NO: 14 - protein, 5 aa, Artificial
  Synthetic peptide
QYSAD

SEQ ID NO: 15 - protein, 5 aa, Artificial
  Synthetic peptide
QYTAD

SEQ ID NO: 16 - protein, 5 aa, Artificial
  Synthetic peptide
QYYAD

SEQ ID NO: 17 - protein, 5 aa, Artificial
  Synthetic peptide
QYNGD

SEQ ID NO: 18 - protein, 5 aa, Artificial
  Synthetic peptide
QYNVD

SEQ ID NO: 19 - protein, 5 aa, Artificial
  Synthetic peptide
QYNID

SEQ ID NO: 20 - protein, 5 aa, Artificial
  Synthetic peptide
QYNLD

SEQ ID NO: 21 - protein, 5 aa, Artificial
  Synthetic peptide
QYNAE

(19) United States
(12) Patent Application Publication
(10) Pub. No.: US 2003/0220232 A1
(43) Pub. Date: Nov. 27, 2003

(54) Peptide and nucleic acid coding therefor for the detection, diagnosis and therapy of diseases of the nervous system

(76) Inventors: Heinrich Brinkmeier, Langenau (DE); Reinhardt Rudel, Ulm (DE); Peter Schneider, Stadtbergen (DE); Andrea Dankwardt, Augsburg (DE)

Correspondence Address: GREENLEE WINNER AND SULLIVAN P C, 5370 MANHATTAN CIRCLE, SUITE 201, BOULDER, CO, 80303, US

(21) Appl. No.: 10/223,594

(22) Filed: Aug. 19, 2002

Related U.S. Application Data
(63) Continuation-in-part of application No. PCT/EP01/01850, filed on Feb. 19, 2001.

(30) Foreign Application Priority Data
Feb. 17, 2000 (DE) DE 100 07 234.8

(51) Int. Cl. A61K 38/08; C07K 7/06; A61K 38/14; C07K 9/00

(52) U.S. Cl. 514/8; 514/17; 530/330; 530/322

(57) ABSTRACT

The invention relates to a novel peptide, fusion proteins and polypeptides containing said peptide, test kits and a method for detecting the peptide, coding nucleic acids and pharmaceutical compositions which contain the peptide or are based on the coding nucleic acid. The peptide serves as a marker for diagnostically detecting inflammatory and/or excitation-inhibitory processes and diseases of the nervous system and as a basis for pharmaceutical compositions for treating diseases of the nervous system or for pharmaceutical compositions having an anaesthetic effect.

### CROSS-REFERENCE TO RELATED APPLICATIONS

This application claims priority under 35 USC 120 to PCT/EP01/01850, filed Feb. 19, 2001, which designates the United States, and claims priority under 35 USC 119 to German patent application DE 100 07 234.8, filed Feb. 17, 2002. Both of these applications are incorporated by reference herein in their entirety.

### BACKGROUND OF THE INVENTION

The invention relates to a new peptide, polypeptides and fusion proteins containing the peptide, test kits and methods for the detection of the peptide, nucleic acids coding therefor and pharmaceutical compositions which comprise the peptide or the coding nucleic acid. The invention also relates to various uses of the peptide, processes for its preparation and antibodies directed against the peptide.

Guillain-Barré syndrome (GBS) is regarded as an autoimmune disease of the peripheral nervous system. Progressive weakness of the limbs up to complete paralysis represents a main feature of the disease. Paraesthesia, loss of sensorial perception and disorders of the autonomous nervous system are furthermore often observed. The maximum severity of the symptoms is reached within one to two weeks, in rare cases within four weeks. The symptoms usually retrogress within months, approximately 80% of patients showing no deficiencies or only mild deficiencies which do not restrict movement.

A disease which resembles GBS but is characterized by a chronic course is called chronic inflammatory demyelinizing polyradiculoneuropathy (CIDP). There is as yet no generally applicable definition for CIDP with the exception of the observation that in contrast to GBS, the progressive phase lasts longer than four weeks, often longer than six months, and that deficiencies often remain in the patient. The mechanism which causes the severe paresis with GBS and CIDP possibly includes an immune reaction and inflammation mediated by T lymphocytes, which follows demyelinization of peripheral neurons. This assumption is confirmed by increased amounts of complement compounds and cytokines observed in the serum and cerebrospinal fluid of GBS patients. The process of demyelinization, especially in the region of the nerve roots, is currently regarded as the decisive mechanism in the development of nerve conduction block. One theory is based on a disorder of the blood/cerebrospinal fluid (CSF) barrier as a relatively early important step in the development of the disease. Another theory claims that leaks develop in the blood/CSF barrier as a consequence of the disease and cause the increased protein content in the CSF. At any rate, non-specific serum constituents without direct reference to the immune system could penetrate into the CSF from the blood, cause neuronal or glial dysfunctions and/or modify neuronal activity. An alternative mechanism is a reduced flow rate of the CSF, which could explain the increased protein content of the CSF. This interpretation requires no impairment or modified selectivity of the blood/CSF barrier. Although all the effects mentioned could be of importance for the course of GBS and CIDP, their actual contribution to the symptoms has not yet been clarified. It has not been possible to establish a connection between the increased protein concentrations in the CSF and specific electrophysiological findings or the clinical picture. Factors in the CSF of GBS patients and multiple sclerosis patients which interact with potential-dependent sodium channels have recently been described (Wüz et al., Muscle and Nerve 18 (1995), 772-781). Brinkmeier et al., (Muscle and Nerve 19, (1996), 54-62) report that the factors have a molecular weight of less than three kDa, and under more stringent test conditions of less than one kDa. On the basis of this observation and the fact that the activity of the factors was not substantially reduced even after incubation of CSF with proteases, the authors concluded that the factors were neither antibodies nor cytokines. The stability of the factors to a heat treatment suggests that these are not proteins. It was also possible to rule out heat-unstable and complement-dependent antiexcitatory factors, which are found e.g. in sera of patients with multiple sclerosis.

All the research results known to date do not allow an unambiguous conclusion of the pathogenesis of the two diseases mentioned. In the same way as for GBS and CIDP, it is equally as impossible for an individual cause to be named for the best known disease characterized by demyelinization, multiple sclerosis (MS). MS is also regarded as an autoimmune disease of the nervous system.

In the case of multiple sclerosis, immune reactions against constituents of the myelin sheath are regarded as the cause of the neurological symptoms. The precise triggering factors of MS are indeed still not clarified, but there is agreement that autoimmune reactions against myelin protein in the inflammation focus lead to detachment of the myelin from the axons and to destruction of myelin-forming cells. An impairment of impulse transmission, axonal damage and loss of axons and nerve cells occur as a result.

Diagnosis is currently based on clinico-electrophysiological data, imaging NMR analyses of the brain and general liquor-diagnostic investigations which are not very disease-specific.

Multiple sclerosis cannot currently be cured, but there are various possibilities for alleviating its course. In an acute attack, anti-inflammatory corticosteroids can be successfully employed. Immunosuppressants and immunomodulators (e.g. interferon β) furthermore have a favourable effect on the course of the disease. Interferon β appears to reduce the substrates with intermittent MS. In addition to pharmacotherapeutic arrangements, physiotherapy, i.e. targeted exercising of the musculature and exercising of movement processes, have also proved to be favourable in improving the quality of life of MS patients.

The earlier the diagnosis is made and treatment is started, the more favourable for the prognosis, since damage caused by active inflammation foci cannot be completely reversed.

### SUMMARY OF THE INVENTION

The present invention is therefore based on the object of providing means and routes which can be employed in a targeted manner for diagnosis and/or treatment of inflammatory and/or impulse-inhibiting processes and diseases of the nervous system, in particular for demyelinizing diseases. Another object of the invention is to provide means and methods which allow the earliest possible diagnosis and therefore early treatment.

These objects are achieved according to the invention by a peptide with the sequence Gln-Tyr-Asn-Ala-Asp (SEQ ID NO: 1) and derivatives thereof, the derivatives differing from the original sequence by the addition, substitution, inversion, insertion, deletion and/or synthetic modification of one or more amino acid(s) and having at least 10% of the sodium ion channel-binding capacity of the peptide (SEQ ID NO: 1) and/or at least 50% of the neuroinhibitory activity, or salts or esters thereof.

### BRIEF DESCRIPTION OF THE DRAWINGS

FIG. 1 shows the dose/effect curve of the synthetic peptide Gln-Tyr-Asn-Ala-Asp (SEQ ID NO: 1) on the “steady-state” inactivation curves. The figure shows the average left shift of the h∞ curve plotted against the peptide concentration.

FIG. 2 shows a western blot. Liquor samples from 3 GBS patients were applied in the tracks marked with (GBS), and the liquor sample of an MS patient was applied in the track marked with (MS). A sample from a healthy individual was applied in the track identified with (contr.). A molecular weight standard was plotted in the right-hand track (14, 22, 31, 45, 66, 97 and 200 kDa).

### DETAILED DESCRIPTION OF THE INVENTION

Some terms are explained below in order to clarify how they are to be understood in the connection of the present Application.

The term “polypeptide” as used below in the description means peptides or proteins composed of 6 or more amino acids.

“Neuroinhibitory activity” here means the capacity of a substance for blocking sodium ion channels. Neuroinhibitory activity can be measured in this connection by the inhibition of sodium ion currents through potential-dependent sodium ion channels. (Review article by Lehmann-Horn et al. in Rev. Physiol. Biochem. Pharmacol. 128; (1996), 198-268). The experimental conditions for measurement of the neuroinhibitory activity are described, for example, in Brinkmeier et al., Muscle and Nerve 19 (1996), 54-62. Neuroinhibitory activity can be determined using differentiated NH15-CA2 [neuroblastoma×glioma] cells (Hamprecht et al., Meth. Enzymol. 109 (1985), 316-41; Brinkmeier et al., Muscle Nerve 19 (1996), 54-62). Example 8 shows the measurement of neuroinhibitory activity.

“Sodium ion channel-binding capacity” here means the capacity of substances for binding to sodium ion channels, in particular potential-dependent sodium ion channels. The sodium ion channel-binding capacity can be determined, for example, as described in Trainer et al., J. Biol. Chem. 271 (1996), 11261-11267. Example 9 shows the measurement of the sodium ion channel-binding capacity by way of example.

The term “homology,” known to one skilled in the art, describes the degree of connection between two or more peptides or polypeptides, which can be determined by the agreement between the sequences by means of known methods, e.g. computer-assisted sequence comparisons (Basic local alignment search tool, S. F. Altschul et al., J. Mol. Biol. 215 (1990), 403-410). The percentage “homology” is given by the percentage of identical regions in two or more sequences, taking into account gaps or other sequence peculiarities. As a rule, special computer programs with algorithms which take account of the particular requirements are employed.

Preferred methods for determination of homology initially generate the greatest agreement between the sequences investigated. Computer programs for determination of homology between two sequences include, but are not restricted to, the GCG program package, including GAP (Devereux, J., et al., Nucleic Acids Research 12 (12): 387 (1984); Genetics Computer Group University of Wisconsin, Madison, (Wis.)); BLASTP, BLASTN and FASTA (Altschul, S. et al., J. Mol. Biol. 215:403-410) (1999)). The BLASTX program can be obtained from the National Centre for Biotechnology Information (NCBI) and from further sources (BLAST Handbuch [BLAST Handbook], Altschul S., et al., NCB NLM NIH Bethesda Md. 20894; Altschul, S., et al., Mol. Biol. 215:403-410 (1990)). The known Smith Waterman algorithm can also be used for determination of homologies.

Preferred parameters for amino acid sequence comparison include the following:

| | |
|---|---|
| Algorithm: | Needleman and Wunsch, J. Mol. |
| | Biol 48:443-453 (1970) |
| Comparison matrix: | BLOSUM 62 from Henikoff and Henikoff, |
| | PNAS USA 89 (1992), 10915-10919 |
| Gap penalty: | 12 |
| Gap length penalty: | 4 |
| Homology threshold | 0 |
| (threshold of similarity): | |

The GAP program is also suitable for use with the above parameters. The above parameters are the default parameters for amino acid sequence comparisons, where gaps at the ends do not reduce the homology value. With very short sequences compared with the reference sequence, it may furthermore be necessary to increase the expectation value to 100,000, and if appropriate to reduce the word size down to 2.

Further examples of algorithms, gap opening penalties, gap extension penalties and comparison matrices, including those mentioned in the program handbook of the Wisconsin Package, version 9, September 1997, can be used. The choice will depend on the comparison to be carried out and furthermore on whether the comparison is carried out between sequence pairs, where GAP or Best Fit are preferred, or between one sequence and a comprehensive sequence databank, where FASTA or BLAST are preferred.

An agreement of 60% determined with the abovementioned algorithm is described as 60% homology in the context of this Application. The same applies to higher degrees of homology.

“Cloning” is intended to mean all the cloning methods known in the prior art which could be employed here but are not all described in detail because they belong to the obvious tools of one skilled in the art.

“Recombinant expression in a suitable host cell” is to be understood as meaning all the expression methods which are known in the prior art in known expression systems and which could be employed here, but are not all described in detail because they belong to the obvious tools of one skilled in the art.

Surprisingly, it has now been found that a peptide with the sequence Gln-Tyr-Asn-Ala-Asp (SEQ ID NO: 1) occurs in the cerebrospinal fluid of patients with multiple sclerosis and Guillain-Barré syndrome. The peptide binds to sodium ion channels and blocks their sodium currents. In a comparison with the known local anaesthetic lidocaine, it is found that the electrophysiological action of a 10 μM solution of the peptide according to the invention corresponds to the action of a 50 μM lidocaine solution on neuronal sodium channels. The importance of functioning sodium ion channels with MS also manifests itself in that the administration of low doses of lidocaine to MS patients with subclinical demyelinizing lesions causes neurological symptoms at plasma levels of 2.7 μg/ml (10 μM) (Sakurai et al., Neurology 42 (1992), 2088-2093). The peptide is thus a significantly more potent local anaesthetic than the lidocaine used therapeutically to date.

The peptide advantageously shows a saturation of the neuroinhibitory effect at a concentration of 100 μM, so that even in the case of overdose in the context of therapeutic use a normalization of axonal activity, but no blocking, is to be expected.

It has furthermore been found that the peptide occurs in the cerebrospinal fluid samples from GBS and MS patients in a concentration range of 8 to 25 μM, but was not to be observed in the controls of healthy individuals. Without being tied to a theory, the peptide could further deteriorate the neurological symptoms of demyelinizing diseases by its binding to the sodium ion channels in the Ranvier constriction rings and an increased inhibition of impulse conduction, especially in neurons which are already affected by the demyelinization.

Antibodies which bind specifically to the peptide have furthermore been found in the serum of a GBS patient. No peptide-specific antibodies were to be detected in sera of healthy control persons. The peptide thus has a high potential for diagnosis of neurological diseases.

The invention thus relates to a peptide with the sequence Gln-Tyr-Asn-Ala-Asp (SEQ ID NO:1) and derivatives thereof which differ from the original sequence by the addition, substitution, inversion, insertion and/or deletion of one or more amino acid(s) and have at least 10%, preferably 50%, particularly preferably 90% of the sodium ion-binding capacity of the peptide (SEQ ID NO:1) and/or at least 50%, preferably 80%, particularly preferably 90% of the neuroinhibitory activity. Derivatives which have an increased neuroinhibitory activity are of interest as active anaesthetics. On the basis of their property of suppressing the electrical excitability of axons and nerve cells, these derivatives are furthermore suitable as neuroprotective agents. According to a preferred embodiment, derivatives with neuroprotective properties are provided for treatment of polyneuropathies, in particular diabetic polyneuropathy, multiple sclerosis (MS), apoplexy and states of pain.

Derivatives which bind to sodium ion channels but have no or only a slight neuroinbibitory activity act as antagonists of the peptide occurring in the cerebrospinal fluid in respect of the sodium ion channel and are thus of great therapeutic importance.

In a preferred embodiment, the derivative is at least 60% homologous, preferably at least 80% and particularly preferably at least 90% homologous to the original sequence (SEQ ID NO:1). The derivative is particularly preferably obtained by conservative substitution of at least one or more amino acid(s) of the peptide (SEQ ID NO:1). It is furthermore preferred here for aspartic acid or glutamic acid to be provided as the C-terminal amino acid.

Conservative modifications are understood as meaning those which are based on the exchange of amino acids and have the lowest possible influence on the (spatial) structure of the peptide. A distinction is made in principle between four physico-chemical groups into which the naturally occurring amino acids are divided. Arginine, lysine and histidine belong to the group of basic amino acids. Glutamic acid and aspartic acid belong to the group of acid amino acids. The non-charged/polar amino acids include glutamine, asparagine, serine, threonine and tyrosine. The non-polar amino acids include phenylalanine, tryptophan, cysteine, glycine, alanine, valine, isoleucine, leucine and proline. A conservative substitution in this connection means the exchange of a given amino acid for an amino acid which belongs to the same physico-chemical group.

Particularly preferred derivatives of the peptide are chosen from: NYNAD (SEQ ID NO:5), SYNAD (SEQ ID NO:6), TYNAD (SEQ ID NO:7), YYNAD (SEQ ID NO:8), QQNAD (SEQ ID NO:9), QNNAD (SEQ ID NO:10), QSNAD (SEQ ID NO:11), QTNAD (SEQ ID NO:12), QYQAD (SEQ ID NO:13), QYSAD (SEQ ID NO:14), QYTAD (SEQ ID NO:15), QYYAD (SEQ ID NO:16), QYNGD (SEQ ID NO:17), QYNVD (SEQ ID NO:18), QYNID (SEQ ID NO:19), QYNLD (SEQ ID NO:20) and QYNAE (SEQ ID NO:21).

These derivatives are derived by conservative substitution of an amino acid from the amino acid sequence (SEQ ID NO: 1) of the peptide according to the invention and therefore have agonistic properties. The invention also contemplates a peptide derived from SEQ ID NO.1 having more than one possible modification. For example, a combination of conservative substitutions can yield an agonistic derivative. Alternatively, a combination of modifications can comprise one conservative substitution and one synthetic modification to yield an agnositic derivative, etc.

In a further preferred embodiment, the peptide is furthermore modified at least on one N-terminal, internal and/or C-terminal amino acid. Such modifications can be of a type such that the peptide structure is lengthened or modified at the C-terminal group and/or lengthened or modified at the N-terminal group and/or lengthened or modified at an internal group/residue position, and/or has corresponding lengthenings and/or modifications at multiple groups. These modifications can be modifications which occur naturally in the liquor or serum; however, the peptide can also be modified synthetically, so that, for example, it is adapted functionally to a diagnostic system. This is the case, for example, if amino acids or other chemical structures function as spacers in order to be able to be recognized by an antibody in an optimum manner as far as possible in a diagnostic test system, after coupling to a carrier molecule, or to show a particular suitability for recognition of the peptide in presentation as an antigen.

Further modifications according to the invention comprise acetylation, glycosylation and/or amidation of N-terminal amino groups, side chain groups and/or C-terminal carboxyl groups. Further modifications comprise conventional N-terminal protective groups, such as the benzyloxycarbonyl group, and/or C-terminal protective groups.

The salts of the peptide are furthermore provided according to the invention. Physiologically tolerated salts, such as e.g. sodium salts, potassium salts, magnesium salts, bicarbonate salts, acetates, citrates and chlorides, are preferred here. The esters of the peptide according to the invention are furthermore included according to the invention, esters which can be cleaved under physiological conditions being preferred. Such esters can have advantages in the galenical formulation and an increased storage stability.

In a further embodiment, the invention provides a polypeptide which comprises at least one peptide according to the invention. This includes both naturally occurring polypeptides, particularly preferably the precursor protein, or fragment thereof, from which the peptide according to the invention originates by cleavage, and polypeptides which are not naturally occurring, which can be obtained by chemical and/or enzymatic synthesis or by genetic engineering processes. If genetic engineering processes are used for the preparation of the polypeptide, all the purification processes known to one skilled in the art, including chromatographic processes, such as ion exchanger, hydrophobic interaction, gel filtration and affinity chromatography, can be used.

It has been found by experiment that a peptide with the sequence Gln-Tyr-Asn-Asp-Ala (SEQ ID NO:2), which thus has an inversion of the last two amino acids compared with the peptide according to the invention, does not have a neuroinhibitory activity in respect of sodium ion channels. This means that the two terminal carboxyl groups of the amino acids aspartic acid are possibly essential for the neuroinhibitory activity of the peptide. Thus, according to one embodiment, those polypeptides which are characterized in that the peptide forms the C terminus of the polypeptide are preferred.

The polypeptide is preferably the natural precursor protein from which the peptide according to the invention with the sequence Gln-Tyr-Asn-Ala-Asp originates by posttranslational modification, in particular processing processes.

According to another aspect, the invention provides a polypeptide chosen from polypeptides with a molecular weight of about 25 kDa, about 35 kDa, about 50 kDa, about 60 kDa, about 80 kDa and about 150 kDa according to SDS polyacrylamide gel electrophoresis under reducing conditions, the polypeptide reacting specifically with a QYNAD-specific antiserum. The size data given relatively by “about” means here that deviations of 0-5%, at most 10%, can result, depending on the buffer system and the size markers chosen.

Six protein bands with molecular weights of about 25 kDa, about 35 kDa, about 50 kDa, about 60 kDa, about 80 kDa and about 150 kDa which showed a specific reaction with a peptide (SEQ ID NO:11)-specific antiserum from rabbits were identified by experiment by western blot analysis of serum or liquor samples from GBS and MS patients. It is to be assumed that the polypeptides are present in solution in the serum and liquor, a higher concentration being observed in the serum than in the liquor. With the exception of the polypeptide of about 35 kDa, the polypeptides in samples from MS and GBS patients were also found in a lower concentration in samples from healthy controls. The 25 and the 35 kDa polypeptide was found in individual patient sera, but not in all.

In a preferred embodiment, each of the polypeptides is obtainable from human liquor or serum. The liquor or the serum particularly preferably originates from a GBS or MS patient.

The polypeptides can be purified in a manner known per se, including chromatographic techniques, such as gel filtration, ion exchanger, hydrophobic interaction and affinity chromatography. The use of antibodies is particularly suitable for affinity chromatography. The use of polyclonal antibodies which recognize the peptide QYNAD specifically is possible for purification of the polypeptides, but the use of monoclonal antibodies is preferred. After the immunoaffinity chromatography, the polypeptides can be separated according to their size. The sequence analysis of the purified polypeptides can be carried out with commercially obtainable, automated protein sequencing machines. A partial sequence can be used with conventional reverse cloning techniques to obtain the entire sequence for a polypeptide and corresponding gene/nucleic acid sequence.

The invention furthermore provides derivatives of the polypeptides, the derivatives differing from the polypeptide by the addition, substitution, inversion, insertion and/or deletion of one or more amino acid(s).

In a preferred embodiment, the derivatives bind to a QYNAD-specific antiserum. The antiserum here can be prepared in a manner known per se by immunizing test animals, such as mice or rabbits. The detection of the binding of the derivative of the polypeptide to the QYNAD-specific antiserum can be carried out, for example, in ELISA. Derivatives which differ from the original sequence by the addition, substitution, inversion, insertion, synthetic modification, and/or deletion of one or more amino acid(s) and have at least 50%, preferably 80%, in particular 90% of the binding capacity of the polypeptide to QYNAD-specific antiserum are included according to the invention.

Preferably, the derivative of the polypeptide is at least 80% homologous to the polypeptide.

It is furthermore preferable for the derivative to be obtained by conservative substitution of one or more amino acid(s) of the polypeptide.

The determination of homology and the definition of conservative substitutions are made here as described above.

The precursor protein(s) is/are furthermore obtainable by searching a human cDNA library from the brain, spinal marrow, lymphocytes, macrophages, oligodendrocytes or glia cells with a probe which has been prepared on the basis of the sequence according to the invention. Suitable cDNA libraries are commercially obtainable, and their production moreover belongs to one skilled in the art; cf. Maniatis et al., Molecular Cloning, Cold Spring Harbor Laboratory Press 1989. The sequence of the oligonucleotide which is suitable as a probe for searching the cDNA library results on the basis of the universal genetic code. However, oligonucleotide sequences which correspond to the frequency of human codon usage are preferred here.

The amino acid glutamine is generally coded by the codons CAA or CAG, preferably on the basis of human codon usage by CAG. The amino acid tyrosine is generally coded by TAT or TAC, preferably by TAC. The amino acid asparagine is coded by AAT or AAC, preferably by AAC. The amino acid alanine is coded by GCT, GCC, GCA or GCG, preferably by GCC. The amino acid aspartic acid is coded by GAT or GAC, preferably by GAC. The codon use reproduced in this connection can be found, for example, in the following literature references: Grantham, R. et al., Nucleic Acids Res. 8 (1980), r49-r62; or the current internet address FTP;//ftp.es.embnet.org/pub/databases/codonusage/hum.cod.

It thus emerges that the probe suitable for searching the cDNA library has the general sequence: CARTAYAAYGCNGAY (SEQ ID NO:3). R here denotes A or G; Y denotes T or C; and N denotes A, G, C or T.

The probe preferably has the sequence CAGTACAACGCCGAC (SEQ ID NO:4). The complement of the abovementioned sequences is also suitable for searching the cDNA library. The oligonucleotide can be prepared with the aid of known solid phase synthesis techniques. To be used as a probe, the oligonucleotide is marked radioactively or non-radioactively (e.g. by fluorescence) with the aid of known processes. The probe is brought into contact with the cDNA library in accordance with processes known to one skilled in the art (Maniatis et al., above). It is preferably brought into contact under stringent conditions; stringent conditions in this connection are incubation at 68° C. overnight in 0.5×SSC; 1% blocking reagent (Boehringer Mannheim), 0.1% sodium lauryl sarcosinate, followed by washing with 2×SSC, 0.1% SDS. The cDNA clones obtained in this manner are isolated and sequenced using known processes. The amino acid sequence of the precursor protein can be derived directly from the nucleotide sequence in a manner known to one skilled in the art. The polypeptide or precursor protein according to the invention can be prepared by chemical and/or enzymatic synthesis or by genetic engineering processes, in particular recombinant expression in heterologous expression systems.

In a further embodiment, variants of the precursor proteins, which are also called polypeptides in this connection, which have neuroinhibitory activity and/or bind to a sodium ion channel are furthermore provided. The sodium ion channel is preferably a potential-dependent sodium ion channel. The neuroinhibitory activity is preferably inhibition of a sodium ion channel. The determination of the binding to a sodium ion channel or of the neuroinhibitory activity can be carried out as above for the peptide according to the invention. In a preferred embodiment, the polypeptides according to the invention have 10%, preferably 50%, particularly preferably 90% of the sodium ion channel-binding capacity of the peptide with the sequence (SEQ ID NO: 1) and/or at least 50%, preferably 80% and particularly preferably 90% of the neuroinhibitory activity.

In a further embodiment, the invention provides a fusion protein which contains at least one peptide and/or polypeptide according to the invention and at least one biologically active polypeptide or an active fragment thereof. In this connection, the term “biologically active polypeptide” is intended to mean all peptides or proteins with biological activity. It is preferable for the biological activity to be an activity which participates in the development and regeneration of cells, tissues and organs of the human and animal body. It is preferable for the biological activity to influence the development and differentiation of cells of the peripheral and central nervous system and supply cells thereof. The fusion protein according to the invention fulfils the task here of increasing the local concentration of the biologically active polypeptide in the vicinity of sodium ion channels, preferably potential-dependent sodium ion channels, on the basis of the peptide or polypeptide sequence according to the invention furthermore contained in the fusion protein. This has the result that biologically active polypeptides which themselves have only a low or moderate binding capacity for these sodium ion channels are significantly increased in their binding capacity. Preferred fusion proteins include ciliary neurotrophic factor (CNTF), “brain-derived” neurotrophic factor (BDNF), neurotrophin 3 (NT-3), neurotrophin 4/5 (NT-4/5) and glia cell-derived neurotrophic factor (GDNF), in each case in combination with the peptide according to the invention.

The term “fusion protein” in this connection means that at least one peptide and/or polypeptide according to the invention is added on to the amino acid sequence of the biologically active polypeptide or an active fragment thereof, and/or is inserted into the amino acid sequence of the biologically active polypeptide, and/or at least one oligopeptide sequence which occurs naturally in the amino acid sequence of the biologically active polypeptide is substituted by a peptide or polypeptide according to the invention. A fusion protein can be generated through recombinant technology as is known in the art.

It is furthermore preferable for the peptide, polypeptide or fusion protein according to the invention also to include on the N terminus a sequence which is relevant for recombinant expression, wherein the sequence relevant for recombinant expression is M or MX, and M denotes methionine and X denotes one or more amino acids as desired. MX can represent, for example, a signal sequence known to one skilled in the art for numerous prokaryotic and eukaryotic proteins. X comprises 1 to 40, preferably 5 to 30 or 15 to 25 amino acids.

The invention furthermore provides nucleic acid molecules which comprise a nucleic acid which codes for a peptide, polypeptide or fusion protein according to the invention.

The nucleic acid contained in the nucleic acid molecule according to the invention can be genomic DNA or synthetic DNA, synthetic DNA also being understood as DNA which contains modified internucleoside bonds. The nucleic acids can furthermore be RNA molecules, which may be necessary e.g. for expression by means of recombinant RNA vector systems.

A nucleic acid molecule which comprises a nucleic acid which is chosen from:

(i) a nucleic acid which codes for a peptide, polypeptide or fusion protein according to the invention;

(ii) a nucleic acid complementary to the nucleic acid according to (i); and

(iii) a nucleic acid which hybridizes with a nucleic acid according to (i) or (ii) and codes for a polypeptide which binds to a sodium ion channel and/or has neuroinhibitory activity,

is provided according to the invention.

The nucleic acids according to (iii) are obtainable, for example, by using a detectably marked probe which corresponds to a nucleic acid according to (i) or (ii) for searching cDNA or genomic DNA libraries. cDNA/genomic DNA libraries from vertebrates, preferably from mammals, and particularly preferably from humans, can generally be used here. The mRNA on which the cDNA library is based is preferably to be obtained from the brain, spinal marrow, lymphocytes, macrophages, oligodendrocytes or glia cells. Positive cDNA/genomic DNA clones are identified in accordance with standard methods; cf. Maniatis et al., above.

In a preferred embodiment, the hybridization described under (iii) is carried out under stringent conditions. Stringent hybridization conditions are e.g. incubation at 68° C. overnight in 0.5×SSC; 1% blocking reagent (Boehringer Mannheim); 0.1% sodium lauryl sarcosinate; followed by washing with 2×SSC; 0.1% SDS.

In a preferred embodiment, the nucleic acid molecule according to the invention comprises a promoter which is suitable for expression, the nucleic acid sequence being under the control of the promoter. The choice of promoter depends on the expression system used for the expression. Constitutive promoters are generally preferred, but inducible promoters, such as e.g. the metallothionein promoter, are also possible.

In a further embodiment, vectors which contain the nucleic acid molecule according to the invention are provided. Numerous cloning and expression vectors are known in the prior art, cf. Recombinant Gene Expression Protocols, Meth. Mol. Biol. vol. 62, Humana Press, New Jersey, USA. The vector used should contain a replication origin and optionally further regulatory regions. The vector can be chosen from bacteriophages, such as λ derivatives, adenoviruses, vaccinia viruses, baculoviruses, SV40 virus, retroviruses, plasmids, such as Ti plasmids from Agrobacterium tumefaciens, YAC vectors and BAC vectors.

The invention furthermore provides host cells which contain the nucleic acid molecule or the vector and are suitable for expression of the nucleic acid molecule. Numerous prokaryotic and eukaryotic expression systems are known in the prior art, the host cells being chosen, for example, from prokaryotic cells, such as E. coli or B. subtilis, from eukaryotic cells, such as yeast cells, plant cells, insect cells and mammalian cells, e.g. CHO cells, COS cells or HeLa cells, and derivatives thereof. Certain CHO production lines in which the glycosylation patterns are modified compared with CHO cells are known, for example, in the prior art.

The present invention furthermore relates to a process for the preparation of the peptide, polypeptide or fusion protein, which comprises culturing of a host cell under conditions which are suitable for expression and optionally purification of the peptide, polypeptide or fusion protein expressed.

Alternatively, the peptides, polypeptides and fusion proteins according to the invention can also be obtained by chemical and enzymatic synthesis, such as, for example, Merrifield synthesis, and/or fragment condensation. Combinations of chemical, enzymatic and recombinant preparation processes are also possible here.

The invention furthermore provides reagents which are specific for the peptides and/or polypeptides according to the invention. Examples of such specific reagents are antibodies, antibody fragments, e.g. Fv, Fab or F(ab)₂ fragments, or antibody derivatives. The antibodies, antibody fragment, e.g. Fv, Fab or F(ab)₂ fragments, or antibody derivatives can be of monoclonal or polyclonal origin. Specific antibodies are generally obtainable by immunizing test animals, such as e.g. mice or rabbits, with the peptides or polypeptides which are preferably coupled to suitable high molecular weight carrier molecules (often proteins), or fusion proteins according to the invention. The immunization can be facilitated here by addition of suitable adjuvants, which are known in the prior art. Monoclonal antibodies are conventionally obtainable by fusion of spleen cells, which have been taken from an immunized mouse, with tumour cells and selection of the hybridomas thereby formed. Those hybridomas which secrete specific antibodies efficiently can be determined here by searching the supernatant. Alternatively, antibodies can be prepared by recombinant processes; in the preparation of recombinant antibodies, the mRNA which functions as the basis for the synthesis of the corresponding cDNA is isolated from hybridoma cells or B lymphocytes and amplified via a PCR. After ligation in a suitable vector and introduction into a suitable host cell culture, the antibody can be isolated from the cell culture supernatants or the cell lysates. Recombinant antibodies allow “humanization” of the antibody and are less immunogenic as a result. The processes in this respect are known in the prior art.

Further reagents which are specific for a peptide or polypeptide according to the invention are sodium ion channel proteins, preferably potential-dependent sodium ion channel proteins and peptide- and/or polypeptide-specific fragments thereof. Potential-controlled human sodium channels are e.g. CIN1_HUMAN, P35498, CIN2_HUMAN, P99250, CIN4_HUMAN, P35499, CIN5_HUMAN, P14524, CIN6_HUMAN, P01118 from the databank under the internet address http://www.expasy.ch/sprot-top.html.

The invention furthermore provides test kits which comprise peptide- or polypeptide-specific reagents. The test kit can furthermore comprise components which are necessary for carrying out detection methods or buffer substances for appropriate dilution and pH adjustment of the peptide- and/or polypeptide-specific reagent. The test kit is particularly suitable for diagnostic purposes. The test kit according to the invention is used for determination (quantitative or qualitative) of the peptide or polypeptide according to the invention, preferably in a body fluid originating from the human body. However, determinations can also be carried out for diagnostic purposes with a body fluid originating from an animal body.

The invention furthermore includes the use of the reagents which are specific for a peptide or polypeptide according to the invention in methods for detection of the peptide and/or polypeptide in a body fluid. The body fluid is preferably chosen from cerebrospinal fluid or blood or blood products or blood constituents.

A method for the detection of the peptide and/or polypeptide according to the invention which comprises carrying out at least one chromatographic process, such as high performance liquid chromatography (HPLC) and/or affinity chromatography is furthermore provided. High performance liquid chromatography, especially if reverse phases are used, is outstandingly suitable for separating off relatively short peptides or polypeptides. The use of columns of small diameter is particularly advantageous here. The affinity chromatography is carried out using the abovementioned antibody, which is specific for the peptide and/or polypeptide. Affinity chromatography is distinguished by its particularly high specificity.

The invention furthermore provides test kits which are suitable for detection of an antibody which is specific for the peptide and/or polypeptide according to the invention, wherein the test kit comprises at least one peptide and/or polypeptide according to the invention. The test kit can furthermore comprise buffer substances which are known in the prior art and are suitable for use of the test kit in determination and diagnostic methods.

The invention furthermore includes the use of the peptide and/or polypeptide in methods for the determination of peptide- and/or polypeptide-specific autoantibodies in a body fluid. The body fluid is preferably chosen from cerebrospinal fluid or blood or blood products. The detection of autoantibodies in the body fluids mentioned is of particular interest, since it can be used as a marker for the demyelinizing diseases which are supposedly mediated by the peptide. Advantageous methods for determination purposes here are immunoassays, ELISA, RIA, membrane-bound test strips, receptor-binding tests or biosensory determinations, the procedures of which are known to one skilled in the art. In the detection of autoantibodies, the peptide, the polypeptide or suitable peptide conjugates are to be used for specific binding of the autoantibodies. In an ELISA detection, for example, the peptide would be immobilized on a microtitre plate. In the test, the specific autoantibodies bind and are then converted into a signal by appropriately marked anti-immunoglobulin antibodies by known processes.

The invention furthermore provides test kits for detection of the nucleic acid which codes the peptides and/or polypeptides according to the invention. The test kits in this case comprise at least one nucleic acid molecule according to the invention, and the nucleic acid molecule preferably comprises the sequence (SEQ ID NO:3) and particularly preferably the sequence (SEQ ID NO:4).

According to the invention, the nucleic acid molecules are used in methods for detection, in a biological sample, of a nucleic acid which codes the peptide and/or polypeptide according to the invention, the method comprising bringing the sample into contact with a nucleic acid molecule which carries a detectable marker and detecting the marker. Hybridization processes can be employed here, and furthermore optionally northern blot and southern blot processes. The detection of a radioactive marker of the nucleic acid molecule can be carried out here by autoradiography in a simple manner.

The invention furthermore provides a process for removing the peptide or polypeptide from a body fluid. The peptide or polypeptide can generally be removed by physical, chemical or biological processes, on the basis of the knowledge of its molecular structure. These processes can be, for example, ultra- or diafiltration. In a preferred embodiment, the process comprises bringing the body fluid into contact with a reagent which is specific for the peptide or polypeptide and removing the complex which contains the peptide or polypeptide and the specific reagent. Preferred specific reagents here are antibodies or sodium ion proteins and specific fragments thereof. It is particularly preferable for the peptide- or polypeptide-specific reagent to be bound to a solid matrix and for the process to comprise adsorption of the peptide or polypeptide on to the matrix. Immunoadsorption processes, which are characterized by a high efficiency of the removal, are of interest in particular here.

In a further embodiment, pharmaceutical compositions which comprise at least one peptide, polypeptide and/or fusion protein according to the invention and optionally a pharmacologically tolerated carrier and/or diluent are provided. Suitable carriers and/or diluents are known in the prior art. The pharmaceutical compositions are preferably suitable for administration in a therapeutically effective manner, for example, intravenous, subcutaneous or intramuscular administration. Alternatively, the pharmaceutical composition can be in the form of an aerosol using suitable aerosol-stabilizing compounds.

It was found in test experiments that the peptide has a potent neuroinhibitory activity. The neuroinhibitory activity observed was significantly higher than the neuroinhibitory activity of the generally used local anaesthetic lidocaine.

The use of the pharmaceutical compositions as an anaesthetic is thus also proposed according to the invention.

For axons and whole nerve cells, in crisis situations such as exist with demyelinization it can be neuroprotective if their electrical excitability is suppressed. Depolarization of neurons or axons often occurs in crisis situations. This can have the consequence of an inflow of sodium and secondarily also of calcium. An increased intracellular calcium accumulation is known to have a neurotoxic effect.

Without being tied to a theory, the increase in the QYNAD concentration observed in MS and GBS patients could have a protective function for axons and therefore also neurons.

The peptide QYNAD has the effect here of normalization of the cation inflow by inhibiting sodium ion inflow.

In experiments, the saturation of the peptide effect was at a concentration of 100 μM, which is advantageous for therapeutic use since it normalizes axonal activity but does not have a blocking action.

The use of the peptide for neuroprotection is thus proposed according to the invention.

The use of the peptide for treatment of polyneuropathies, in particular diabetic polyneuropathy, is proposed according to the invention. Polyneuropathies are damage to the peripheral nerves.

The use of the peptide for treatment of multiple sclerosis (MS) is furthermore proposed. The possible mechanism here could be inhibition of axonal degeneration such as occurs in some MS patients. The peptide could penetrate into demyelinizing foci via the vascular system and exert protective actions on the axons there.

The peptide is envisaged for treatment of the consequences of apoplexy. The neuroprotective property of the peptide here should attenuate the neurodegenerative processes in the surroundings of the tissue primarily affected. The use for treatment of states of pain is furthermore proposed. The neuroprotective property here normalizes the function of hyperactive afferent neurons.

As stated above, the peptide according to the invention has been observed in the liquor of MS patients and GBS patients but not in healthy subjects. The two diseases mentioned belong to the demyelinizing diseases which are characterized by a dissolving of the myelin sheaths which surround the nerve cells. Some symptoms of MS indicate that MS is to be regarded as an autoimmune disease. Peptides and polypeptides according to the invention which have an increased binding capacity for sodium ion channels but no or only low neuroinhibitory activity are active as antagonists in respect of the naturally occurring pentapeptide with the sequence SEQ ID NO:1. Pharmaceutical compositions which comprise these peptides and/or polypeptides are thus proposed according to the invention for treatment of the demyelinizing diseases and generally for treatment of autoimmune diseases. Further fields of use would be diagnosis and therapy of neurodegenerative diseases, Alzheimer's disease and amyotrophic lateral sclerosis.

According to the invention, both agonists and antagonists of the peptide with the sequence (SEQ ID NO:1) are thus of therapeutic interest in the treatment of diseases mediated by lymphocytes, preferably autoimmune diseases and allergies.

The potential-dependent sodium ion channels are coded by a multigene family. The various iso forms of potential-dependent sodium ion channels are heterotrimeric proteins which consist of a large, highly glycosylated α-subunit and one or two small β-subunits. Eight different genes (SCN1A to SCN8A) which code for the α-subunit are known so far, most of them being expressed in the brain, peripheral nervous system and muscle. It is furthermore known that certain hereditary diseases are associated with certain sodium ion channel-coding genes. It has thus been found that hyperkaliemic periodic paralysis, a hereditary human muscle disease, is associated with SCN4A. The hereditary diseases paramyotonia congenita and potassium-enhanced myotonia are also associated with SCN4A. Hereditary cardiac arrhythmia shows an association with SCN5A. “Motor end plate disease” in mice is associated with SCN8A. Agonists or antagonists in respect of the peptide of the sequence (SEQ ID NO:1) can thus be used for treatment of hereditary muscle diseases and cardiac arrhythmias. If the pharmaceutical compositions comprise fusion proteins, the use thereof depends on the further biologically active polypeptides on which the fusion protein is based. The biologically active polypeptide is preferably one which influences the development and/or differentiation of cells of the peripheral or central nervous system or the cells which supply them, such as e.g. nerve growth factor (NGF), CNTF, ciliary neurotrophic factor, BDNF, “brain-derived” neurotrophic factor, NT-3, neurotrophin 3, NT-4/5, neurotrophin-4/5 and GDNF, glia cell-derived neurotrophic factor. These pharmaceutical compositions are generally appropriate for treatment of neurodegenerative diseases in which the biologically active polypeptides have only a low affinity for neuronal structures and the concentration thereof can thus be increased significantly by increasing neuronal affinity. In this connection, the treatment of Alzheimer's disease, which is characterized by a progressive degeneration of neuronal structures, is of interest in particular.

The invention furthermore provides pharmaceutical compositions which comprise at least one reagent which is specific for a peptide and/or polypeptide according to the invention and optionally a pharmacologically tolerated carrier and/or diluent. Such specific reagents are preferably chosen from specific antibodies and sodium ion channel proteins or binding fragments thereof. Pharmacologically tolerated carriers and/or diluents are known in the prior art. Peptide- and/or polypeptide-specific reagents which block binding of the peptide or polypeptide to the sodium ion channel can be used for treatment of demyelinizing and neurodegenerative diseases. With suitable labelling of the specific reagents, for example radioactively or non-radioactively, the pharmaceutical compositions can be used as diagnostic agents. These diagnostic agents can also be used in NMR or nuclear magnetic spin tomography processes.

The invention furthermore provides pharmaceutical compositions which comprise at least one nucleic acid molecule according to the invention and optionally a pharmacologically tolerated carrier and/or diluent. These pharmaceutical compositions are suitable for use both in diagnostic and in therapeutic processes. The diagnostic processes here include in situ hybridization. The invention regards as possible therapeutic uses somatic gene therapy, which means suppression of the expression of a disease-mediating gene or the replacement of a defective gene by a correct copy. The processes employed here are, inter alia, anti-sense and sense therapy, suitable vectors and processes being known to one skilled in the art (Weiss et al., Cell Mol. Life Sci 55 (1999), 334-358).

The intention with the following examples is to illustrate the invention but not to limit this in any way. Further embodiments, which are likewise included, are accessible to one skilled in the art on the basis of the description of the examples.

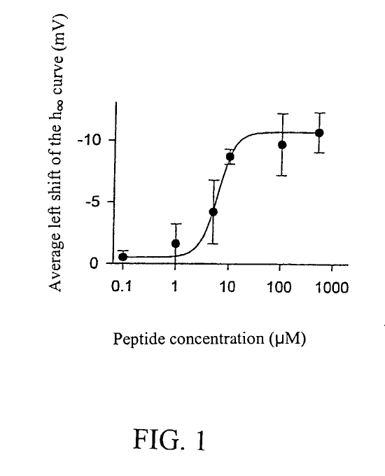

FIG. 1 shows the dose/effect curve of the synthetic peptide Gln-Tyr-Asn-Ala-Asp (SEQ ID NO: 1) on the “steady-state” inactivation curves. The figure shows the average left shift of the h∞ curve plotted against the peptide concentration.

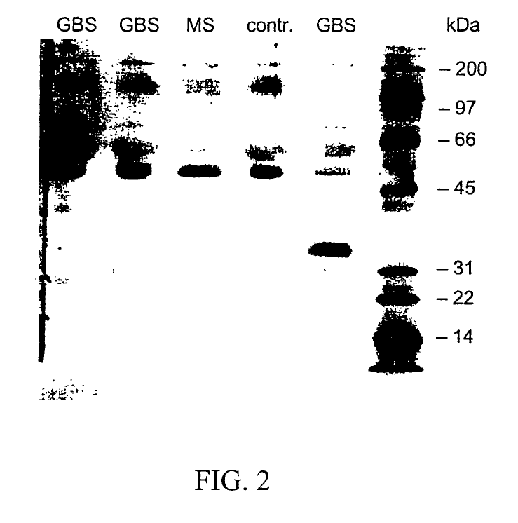

FIG. 2 shows a western blot. Liquor samples from 3 GBS patients were applied in the tracks marked with (GBS), and the liquor sample of an MS patient was applied in the track marked with (MS). A sample from a healthy individual was applied in the track identified with (contr.). A molecular weight standard was plotted in the right-hand track (14, 22, 31, 45, 66, 97 and 200 kDa).

### EXAMPLES

### Example 1

### Use of Detection Methods for the Peptide and Modifications Thereof for Diagnosis

The peptide and modifications or derivatives thereof are used according to the invention as markers for medical diagnosis of diseases, preferably diseases of the nervous systems, such as, for example, GBS or MS in humans. In this function, it can also be used for therapy control. Uses in veterinary medicine are also possible.

The use of the peptide as a marker is preferably carried out in conventional diagnostic methods, such as blotting methods, immunoassays, biosensory methods or comparable methods. In these, the peptide or modifications and derivatives thereof are employed in appropriate binding tests, for example in immunoassays. The peptide here can also be coupled or bound via its C terminus, its N terminus or other suitable functional units to carrier or marker proteins, in particular also enzymes, or also colloids with the aid of known processes, preferably covalently or adsorptively. These conjugates can also be used in the diagnostic methods and represent a constituent of the invention.

By coupling the peptide or its derived structure to proteins or other carrier structures and subsequent immunization with these conjugates, antibodies which specifically recognize the peptide structure and can thus be employed for diagnostic determination of the peptide, for example in immunoassays, can be obtained. These antibodies, which are obtained by immunization with the aid of the peptide, its conjugates or structures derived therefrom, also fall within the scope of the invention. In a diagnostic system which is preferably used, antibodies directed against the peptide are preferably immobilized, for example on adsorbent microtitre plates, by established processes. In a competitive immunoassay, the free peptide structure from the liquor or serum sample competes with a constant amount of added peptide, which is, for example, enzyme-marked, for the binding sites of the antibodies, as a result of which a quantifiable signal is finally achieved for quantitative determination of the peptide. During the preparation of the peptide-protein or peptide-enzyme conjugates, the peptide is conventionally modified, usually by introduction of a spacer arm to the carrier protein, such that an optimum presentation of the peptide is ensured as efficiently as possible. Other conceivable assays with different markers or strategies for presentation of the peptide in the diagnostic system are also a constituent of the invention.

The peptide can also be employed for therapy control in this function.

The first diagnostic method mentioned is indicated for the various types of demyelinizing diseases, such as GBS and multiple sclerosis. By determination of the structures to be analysed in the corresponding body fluids, preferably cerebrospinal fluid or serum, which either relates to the peptide itself or relate to structures which can be present endogenously in the body fluids and bind specifically to the peptide, such as, for example, receptors or autoantibodies, diagnostic conclusions in respect of possible diseases are rendered possible. These conclusions can be connected with the diagnosis for identification of diseases, or can also be used, for example, for monitoring the course of the diseases in question.

The second diagnostic method mentioned relates to applications of the methods connected with the peptide structure for research purposes, where this relates in particular to clarification of molecular processes in connection with physiological processes under pathophysiological aspects in particular. The structure disclosed can also be used as a diagnostic marker in evaluation in connection with the development of medicaments.

### Example 2

### Isolation of Antibodies Against the Peptide for use in the Immunological Test (Diagnostic) Method

The antibodies are obtained by using the peptide or structure derived therefrom, preferably protein-peptide conjugates, for immunization. The peptide or derivatives thereof are conventionally modified, for example with spacers, before coupling thereof to carrier structures (carrier protein), in order to promote a better recognition by the immune system and thus to obtain more efficient antibodies. Antibodies often form the basis of a diagnostic method which renders possible routine detection of peptide structures in diagnostic tests. Human liquor samples in particular, but also human serum or other body fluids of human or animal origin, are used as the matrix for this.

### Example 3

### Detection of Autoantibodies Directed Against the Peptide for Diagnosis of Autoimmune Diseases

The peptide and derived structures thereof are also used according to the invention to detect, in body fluids, antibodies which are directed against the peptide structure. They are used inasmuch as a marker structure for autoimmune diseases, and are employed as the target antigen in diagnostic methods. The diagnostic use of molecules which bind the peptide specifically, for example receptor structures, is moreover also included according to the invention. For this, the peptide, preferably after covalent coupling to a macromolecular carrier, is immobilized adsorptively or directly covalently, for example on activated and commercially available microtitre plates. The liquor or serum samples to be determined are incubated on these surfaces, for example in microtitre plates. Any autoantibodies present which are directed against the peptide are bound and can then be detected and quantified, for example by enzyme-marked anti-human antibodies.

For both Examples 1 and 3, methods of detecting peptides of the invention or peptide-specific reagents such as autoantibodies to peptides of the invention can be performed using amplification strategies known in the art such as the avidin-biotin system.

### Example 4

### Uses of the Peptide for Diagnostic Purposes with Therapeutic Aims

Possible uses consist of, for example, the evaluation of medicaments, where the course of the disease can be determined by means of diagnostic measures based on the peptide, for example in the context of evaluation of active compounds or in clinical studies of new therapeutic agents.

### Example 5

### Uses of the Peptide in Therapy

Aims also consist of eliminating the peptide from the corresponding body fluids by the use of suitable techniques (for example by controlled liquor filtration or the therapeutic use of peptide-specific antibodies). Knowledge of the peptide serves as the basis for controlled elimination thereof by physical, chemical or biological processes for controlled removal from the biological matrix or for suppression of the biological activity of the peptide, for example by limiting or completely suppressing the activity thereof by binding to other molecules, for example to antibodies or receptor molecules.

Another possibility consists of displacing the peptide from the target structures, for example by the use of structurally related peptides in therapy.

### Example 6

### Use of the Coding DNA in the Medical Diagnosis of Diseases of the Nervous System

For diagnostic and therapeutic purposes, the nucleic acid which codes the peptide or derivatives thereof can also be used. Established methods for selective determination of certain nucleic acid sequences, such as DNA probes, are used for diagnosis which goes beyond peptide analysis. The performance of the peptide and nucleic acid diagnosis is employed according to the particular specific test requirements.

### Example 7

### Use of the Coding DNA in the Therapy of Diseases of the Nervous System

Therapeutic applications can be based on knowledge of the nucleic acid sequence. These therapeutic applications can be performed, for example, using anti-sense sequences at the level of the coded RNA and can thus have future application in gene therapy.

### Example 8

### Neuroinhibitory Assay

Determination of the Neuroinhibitory Activity

The neuroinhibitory activity was determined by the inhibition of sodium ion channels using differentiated NH15-CA2 neuroblastoma-x-glioma cells. The culture conditions and morphological and physiological parameters of the differentiation of these cells are known (Hamprecht et al., Meth. Enzymol. 109 (1985), 316-41). For the assay, the cells were transferred into a hydrophobic test dish which was filled with standard external liquid (140 mM NaCl; 3.5 mM KCl; 1.0 mM CaCl₂; 1.0 mM MgCl₂; 2 mM HEPES, pH 7.4). The dish was on the platen of an inverted microscope for observation of the cells, while these were treated with pipettes which were filled with internal solution (140 mM CsCl; 1.4 mM MgCl₂; 10 mM EGTA and 10 mM HEPES (peak resistances: 300 to 500 kΩ)). The sodium currents were induced and recorded in the whole cell mode. The determination of the maximum current amplitudes as a response to repetitive rectangular pulses from −85 to −20 mV lasting 8 ms before, during and after administration of the test solution can be used as a rapid test for the neuroinhibitory activity. The decrease in the current was recorded in three to five cells and the means were determined. An extended test consisted of determination of the “steady-state” activation and inactivation parameters of sodium ion channels. To determine the dependence of the activation on potential, a cyclic pulse program was used, which comprised pre-pulses which depolarized the cells from an HP (holding potential) of −85 mV to −135 mV over 100 ms, and subsequent variable test pulses which depolarized for 8 ms in 4 mV steps from −65 to +31 mV. For the dependence of the inactivation on potential, a similar program was used, which comprised a fixed 100 ms conditioning pulse at −135 mV, a variable 32 ms prepulse in the course of −135 to −19 mV in 4 mV steps and a constant test pulse which depolarized the cells to −20 mV. The size of the left shift of the h curve in the presence of test solutions was used as a measured of the inhibition of the sodium ion channels.

The neuroinhibitory activity of the synthetic peptide with the sequence (SEQ ID NO:1) was determined by the inhibition of the sodium currents by ion channels which were induced by 1 Hz rectangular pulses from 85 to −10 mV. FIG. 1 shows the result of seven measurements which were carried out on NH15-CA2 neuroblastoma-x-glioma cells. In each case the mean with the standard deviation is shown. It was found that a peptide concentration of less than 10 μm gave a half-maximum inhibition of the sodium ion currents. The peptide-mediated blocking of the ion channel was rapid and reversible.

### Example 9

### Determination of the Sodium Ion Channel-binding Capacity

The sodium ion channel-binding capacity of a given peptide, polypeptide or fusion protein can be determined generally by competition with tritylated batrachotoxinin A 20-α-benzoate, [benzoyl-2,5-][³H] BTXB, on purified and reconstituted sodium ion channels (Trainer et al., J. Biol. Chem. 271 (1996), 11261-11267). The binding reactions are started with a four-fold dilution of 50 μl and purified and reconstituted sodium ion channels (5-10 pmol) in standard binding medium (Sharkey et al., Mol. Pharmacol. 31 (1986), 273-278). The reconstituted channels are incubated at 25° C. for 16 hours with [³H] BTXB and where appropriate the peptides, polypeptides or fusion proteins according to the invention. The binding reactions are stopped by addition of choline washing medium. The samples are filtered through GF/F filters (Tamkon et al., J. Biol. Chem. 259 (1984), 1676-1688). The non-specific binding is determined in the presence of 300 μM veratridine.

### Example 10

### Detection of QYNAD-specific Antibodies in Patients

To investigate whether antibodies against the peptide QYNAD (SEQ ID NO:1) occur in the serum of GBS patients, the peptide was coupled C-terminally and covalently to BSA (bovine serum albumin). This conjugate (QYNAD-BSA) was immobilized on a microtitre plate. The microtitre plate was washed. The serum sample of a GBS patient or the controls was subsequently incubated with the immobilized conjugate. The microtitre plate was washed. To detect the binding, the human IgG bound was rendered visible in a secondary reaction (colour reaction) by incubation with a marked anti-human IgG-specific antibody and reading of the plate.

A reproducible and clearly positive reaction was found here, i.e. the patient serum contained peptide-specific antibodies.

Controls: No reaction manifested itself with control sera of 15 healthy blood donors. As a further specificity control, instead of QYNAD-BSA, only the carrier BSA was immobilized on the microtitre plate. The test was otherwise carried out as described above. The use of BSA alone showed no reaction on incubation with the patient serum. This shows a specificity of the patient serum for the peptide QYNAD.

### Example 11

### Isolation and Characterization of the Precursor Protein

a) Preparation of a QYNAD-specific rabbit antiserum and purification of the IgG fraction.

A QYNAD-specific antiserum was prepared in a manner known per se by immunization of rabbits and subsequent isolation of serum. For purification of the IgG fraction, the antiserum was incubated with protein A and the antibodies of the IgG type bound thereto were eluted.

b) Preparation of the samples

All the patient liquors were prepurified by ultrafiltration according to size and the >3,000 Da fraction was used further for the search for the precursor protein.

c) SDS polyacrylamide gel electrophoresis

In each case 10 μg liquor protein were reduced in a Laemmli sample buffer (70 mM SDS, 0.1 mM DTT, pH 6.8) and denatured by heating at 95° C. for 5 minutes.

The samples were applied to a 12% SDS polyacrylamide gel. The separation was carried out in a Tris/glycine running buffer system (Biorad chamber, Minigel) at a constant current strength of 20 mA in the separating gel.

d) Electro-transfer (western blot)

For electro-transfer by the semi-dry transfer process (semi-dry blotting), the Multiphor II™ chamber (Amersham/Pharmacia Biotech) was used. The transfer took place on nitrocellulose membranes (0.45 μm, Protean BA 85, Schleicher und Schuell). A discontinuous buffer system of the following composition was used:

Anode 1: 0.3 M tris

Anode II: 0.1 M tris

Cathode: 0.1 M aminocaproic acid, 0.01% (w/v) SDS

The transfer was carried out at 0.8 mA/cm² over 60 minutes at room temperature.

e) Immunodetection

The nitrocellulose membranes were blocked against non-specific binding by incubation with 5% milk powder in TBS buffer. The QYNAD-specific purified rabbit antibodies were then added in a dilution of 1:2,500 and incubation was carried out for one hour at room temperature. The nitrocellulose membranes were washed several times with TTBS buffer (0.05% v/v) Tween 20™). A biotinylated anti-rabbit IgG-specific antibody (Biorad) was used as the secondary antibody. This was incubated with the nitrocellulose membranes in a dilution of 1:3,000 for one hour at room temperature. The nitrocellulose membranes were subsequently incubated with a streptavidin-alkaline phosphatase conjugate. For the detection, the nitrocellulose membranes were incubated with BCIP/NBT substrate (Biorad), which is converted into an insoluble colour complex as a function of the amount of bound enzyme.

The results are shown in FIG. 2. In this, (GBS) and (MS) denote the application of liquor samples from GBS and MS patients respectively. (contr.) means application of a liquor from a healthy donor. A molecular weight standard was applied in the right-hand track.

Several positive bands were found here in the molecular weight range of about 150 kDa (A), about 80 kDa (B), about 60 kDa (C) and about 50 kDa (D), both in the GBS and MS patient liquors and in the liquor of a healthy donor; cf. tracks 1 to 5. In one case, there was an intense band at 35 kDa (F). The control experiment (test procedure without 1st antibody) was negative.

In some further GBS liquors tested (data not shown), an additional band was observed in the molecular weight range of 25 kDa (E).

Bands A, B, C and D were furthermore observed in a serum of a healthy donor and in a GBS serum (data not shown).

These data prove the assumption that the precursor protein is a protein dissolved naturally in human serum and liquor.

### Example 12

### Generation of Antibodies to QYNAD

Antibodies to QYNAD are generated using a hapten-carrier conjugate or a combination of more than one hapten-carrier conjugate. In the case where more than one hapten-carrier conjugate is used, the combination of conjugates can comprise the same carrier conjugated to the hapten by a different procedure or a different carrier that is conjugated to the hapten by the same or different procedure.

A first hapten-carrier conjugate is prepared where the hapten is QYNAD or another peptide of the invention. An exemplary carrier is keyhole limpet hemocyanin (KLH). Other exemplary carriers include the QYNAD peptide coupled by a glutaraldehyde procedure as is well-known in the art to the carrier, where the coupling occurs via an NH2 group of the peptide, such as the N-terminus or the amino group of the asparagine residue.

A second QYNAD-KLH conjugate is prepared where a bisbenzimide reaction is used by methods known in the art to effect coupling via the OH group of the tyrosine residue of the peptide. A spacer compound can be used to separate the peptide from the carrier.

The first and second conjugates are mixed and used for immunization of rabbits. The resulting polyclonal antibodies are used in an ELISA for detection of QYNAD or a precursor protein.

Those of ordinary skill in the art will appreciate that materials (e.g., conjugates, reagents, adjuvants, etc.), methods and procedures other than those specifically disclosed herein can be employed in the practice of this invention without resort to undue experimentation. All such art-known materials, methods and procedures are encompassed by this invention. Those of ordinary skill in the art will recognize the existence of functional equivalents of materials, methods and procedures disclosed herein. All such art-known functional equivalents are encompassed by this invention. This invention also encompasses functional equivalents that become available to the art in the future and which will be so recognized by those of ordinary skill in the art. All references cited herein are incorporate by reference herein in their entirety.

1. Peptide with the sequence Gln-Tyr-Asn-Ala-Asp (SEQ ID NO:1) and derivatives thereof, the derivatives differing from the original sequence by the addition, substitution, inversion, insertion, deletion and/or natural or synthetic modification of one or more amino acid(s) and having at least 10% of the sodium ion channel-binding capacity of the peptide (SEQ ID NO:1) and/or at least 50% of the neuroinhibitory activity, or salts or esters thereof.

2. Peptide according to claim 1, characterized in that the derivative is at least 80% homologous to the original sequence (SEQ ID NO:1).

3. Peptide according to claim 1 or 2, characterized in that the derivative is obtained by conservative substitution of one or more amino acid(s) of the peptide (SEQ ID NO:1).

4. Peptide according to claim 3, characterized in that the derivative has an amino acid sequence which is chosen from SEQ ID NO:5 to SEQ ID NO:21.

5. Peptide according to one of claims 1 to 4, characterized in that the peptide is furthermore modified at least on one N-terminal, internal and/or C-terminal amino acid.

6. Peptide according to claim 5, characterized in that the modification is an acetylation, glycosylation and/or amidation.

7. Polypeptide comprising at least one peptide according to one of claims 1 to 6.

8. Polypeptide according to claim 7, characterized in that the peptide is on the C terminus of the polypeptide.

9. Polypeptide according to claim 7 or 8, characterized in that the polypeptide binds to a sodium ion channel and/or has neuroinhibitory activity.

10. Polypeptide according to claim 9, characterized in that the neuroinhibitory activity is the inhibition of a sodium ion channel.

11. Polypeptide according to one of claims 9 or 10, characterized in that the polypeptide has at least 10% of the sodium ion channel-binding capacity of the peptide (SEQ ID NO:1) and/or at least 50% of the neuroinhibitory activity.

12. Polypeptide chosen from polypeptides with a molecular weight of about 25 kDa, about 35 kDa, about 50 kDa, about 60 kDa, about 80 kDa and about 150 kDa according to SDS polyacrylamide gel electrophoresis under reducing conditions, the polypeptide reacting specifically with a QYNAD-specific antiserum; and derivatives thereof which differ from the original sequence by the addition, substitution, inversion, insertion and/or deletion of one or more amino acid(s) and have at least 50% of the binding capacity of the polypeptide on QYNAD-specific antiserum.

13. Polypeptide according to claim 12, characterized in that the polypeptide is obtainable from human liquor or serum.

14. Polypeptide according to claim 13, characterized in that the liquor or the serum originates from a GBS or MS patient.

15. Polypeptide according to claim 12, characterized in that the derivative is at least 80% homologous to the original sequence.

16. Polypeptide according to claim 12 or 15, characterized in that the derivative is obtained by conservative substitution of one or more amino acids of the polypeptide.

17. Fusion protein comprising at least one peptide or polypeptide according to one of claims 1 to 16 and at least one biologically active polypeptide, or an active fragment thereof.

18. Nucleic acid molecule comprising a nucleic acid chosen from:
(i) a nucleic acid which codes for a peptide or polypeptide according to one of claims 1 to 11;
(ii) a nucleic acid complementary to the nucleic acid according to (i); and
(iii) a nucleic acid which hybridizes with a nucleic acid according to (i) or (ii) and codes for a polypeptide which binds to a sodium ion channel and/or has neuroinhibitory activity.

19. Nucleic acid molecule according to claim 18, characterized in that the hybridization according to (iii) is carried out under stringent conditions.

20. Nucleic acid molecule according to claim 19, characterized in that stringent conditions are 0.5×SSC at 68° C.

21. Nucleic acid molecule according to one of claims 18 to 20, characterized in that the nucleic acid is genomic DNA, cDNA, synthetic DNA or RNA.

22. Nucleic acid molecule according to one of claims 18 to 21, furthermore comprising a promoter which is suitable for expression control, the nucleic acid which codes for a peptide or polypeptide being under the control of a promoter.

23. Vector comprising a nucleic acid molecule according to one of claims 18 to 22.

24. Host cell containing a nucleic acid molecule according to one of claims 18 to 22 and/or a vector according to claim 24, wherein the host cell is a prokaryotic or eukaryotic cell which is suitable for expression of the nucleic acid molecule.

25. Process for the preparation of a peptide and/or polypeptide according to one of claims 1 to 16, comprising culturing of a host cell according to claim 24 under conditions which are suitable for expression, and optionally purification of the peptide or polypeptide expressed.

26. Antibody which is specific for a peptide or polypeptide according to one of claims 1 to 16.

27. Antibody according to claim 26, characterized in that the antibody, the antibody fragment or antibody derivative is monoclonal, polyclonal or recombinant.

28. Test kit for the detection of a peptide or polypeptide according to one of claims 1 to 16, comprising at least one reagent which is specific for the peptide and/or polypeptide.

29. Test kit according to claim 28, characterized in that the peptide- and/or polypeptide-specific reagent is chosen from an antibody according to claim 26 or 27, a sodium ion channel protein and peptide- and/or polypeptide-specific fragments thereof.

30. In vitro method for the detection of a peptide and/or polypeptide according to one of claims 1 to 16 in a biological sample, comprising bringing the sample into contact with a reagent specific for the peptide and/or polypeptide and detection of the binding.

31. Method for detection of a peptide and/or polypeptide according to one of claims 1 to 16, comprising carrying out at least one chromatographic process, such as high performance liquid chromatography (HPLC) and/or affinity chromatography.

32. Use of a reagent which is specific for a peptide and/or polypeptide according to one of claims 1 to 16 for determination of the peptide and/or polypeptide in a body fluid.

33. Use according to claim 32, characterized in that the body fluid is chosen from cerebrospinal fluid, blood and blood products or blood constituents.

34. Process for removal of a peptide or polypeptide according to one of claims 1 to 16 from a body fluid, comprising bringing the fluid into contact with a reagent which is specific for the peptide or polypeptide and removing the complex formed.

35. Process according to claim 34, characterized in that the peptide- or polypeptide-specific reagent is bound to a solid matrix and the process comprises absorption of the peptide or polypeptide.

36. Test kit for the detection of an antibody which is specific for the peptide and/or polypeptide according to one of claims 1 to 16, comprising at least one peptide and/or polypeptide according to one of claims 1 to 16.

37. In vitro method for the detection of an antibody which is specific for the peptide and/or polypeptide according to one of claims 1 to 16 in a biological sample, comprising bringing the sample into contact with a peptide and/or polypeptide according to one of claims 1 to 16 which carries a detectable marker, and detection of the marker.

38. Use of the peptide and/or polypeptide according to one of claims 1 to 16 for the determination of peptide- and/or polypeptide-specific autoantibodies in a body fluid.

39. Use according to claim 38 for the determination of autoantibodies, characterized in that the body fluid is chosen from cerebrospinal fluid, blood and blood products.

40. Test kit for the detection of a nucleic acid which codes for a peptide and/or polypeptide according to one of claims 1 to 16, comprising at least one nucleic acid molecule according to one of claims 18 to 22.

41. In vitro method for the detection, in a biological sample, of a nucleic acid which codes a peptide and/or polypeptide according to one of claims 1 to 16, comprising bringing the sample into contact with a nucleic acid molecule according to one of claims 18 to 22 which carries a detectable marker; and detecting the marker.

42. Use of a nucleic acid molecule according to one of claims 18 to 22 as a probe for the detection, in a biological sample, of a nucleic acid which codes a peptide and/or polypeptide according to one of claims 1 to 16.

43. Pharmaceutical composition, characterized by at least one peptide and/or polypeptide according to one of claims 1 to 16 and optionally a pharmacologically tolerated carrier and/or diluent.

44. Pharmaceutical composition according to claim 43 for therapeutic use as an anaesthetic.

45. Pharmaceutical composition according to claim 43 for neuroprotection.

46. Pharmaceutical composition according to claim 45 for treatment of a disease chosen from polyneuropathies, in particular diabetic polyneuropathy, and multiple sclerosis.

47. Pharmaceutical composition according to claim 45 for treatment of the consequences of apoplexy or states of pain.

48. Pharmaceutical composition according to claim 43 for therapeutic use for demyelinizing diseases or neurodegenerative diseases.

49. Pharmaceutical composition, characterized by at least one reagent which is specific for a peptide and/or polypeptide according to one of claims 1 to 16 and optionally a pharmacologically tolerated carrier and/or diluent.

50. Pharmaceutical composition according to claim 49 for diagnostic or therapeutic use for demyelinizing or neurodegenerative diseases.

51. Pharmaceutical composition, characterized by at least one nucleic acid molecule according to one of claims 18 to 22 and optionally a pharmacologically tolerated carrier and/or diluent.

52. Pharmaceutical composition according to claim 51 for diagnostic or therapeutic use for demyelinizing or neurodegenerative diseases.
